(19) United States
(12) Patent Application Publication
(10) Pub. No.: US 2012/0128634 A1
(43) Pub. Date: May 24, 2012

(54) USE OF COLLINSELLA AEROFACIENS FOR REDUCING BLOATING

(75) Inventors: Patrick Veiga, Suresnes (FR); Raish Oozeer, Montgeron (FR); Karine Roy, Palaiseau (FR); Stéphanie Cools-Portier, Epinay sur Orge (FR); Aurore Tessier, Lacqueue les Yvelines (FR); Pascale Rondeau, Savigny sur Orge (FR); Denis Guyonnet, Levallois-Perret (FR); Richéle Deodata Wind, Nijmegen (NL)

(73) Assignee: CAMPAGNIE GERVAISE DONONE, Paris (FR)

(21) Appl. No.: 13/318,134

(22) PCT Filed: Apr. 30, 2010
(86) PCT No.: PCT/IB10/01268

§ 371 Date: Feb. 10, 2012

(30) Foreign Application Priority Data
Apr. 30, 2009 (IB) PCT/IB2009/005735

(51) Int. Cl. A61K 35/74; A61P 1/00

(52) U.S. Cl. 424/93.4

(57) ABSTRACT

The invention relates to compositions comprising Collinsella aerofaciens for use for reducing bloating, in particular in subjects suffering from irritable bowel syndrome.

The invention relates to the modulation of intestinal microflora, for treating or preventing gastro-intestinal diseases, in particular of bowel disorders such as irritable bowel syndrome.

Irritable bowel syndrome (IBS) is a common bowel disorder, affecting up to 15% of the Western population. It is characterised by a mixture of symptoms, including abdominal pain or discomfort, bloating, and constipation, diarrhoea, or both. IBS has been sub-classified into IBS with constipation (IBS-C), IBS with diarrhoea (IBS-D), or IBS with alternating constipation or diarrhoea symptoms (IBS-M).

Although several causes, including stress, food intolerances, and an imbalance of the intestinal microflora have been identified, the etiology of IBS is poorly understood. Therefore, at the moment, there is no global cure for this disease, and the treatment is focused on relieving symptoms.

Bloating is an extremely common symptom of irritable bowel syndrome, and is one of the most bothersome.

There is growing evidence that IBS is associated with alterations in the gastro-intestinal microflora (Malinen et al., Am J Gastroenterol, 100, 373-82, 2005; Matto et al., FEMS Immunol Med Microbiol, 43, 213-22, 2005; Maukonen et al., J Med Microbiol, 55, 625-33, 2006; Kassinen et al., Gastroenterology, 133, 24-33, 2007), and ingestion of probiotic bacteria has been reported to result in alleviation of some of the symptoms of IBS (for review cf. for instance Parkes et al., Am J Gastroenterol, 103, 1557-67, 2008).

For instance it has been shown that a fermented dairy product containing the Bifidobacterium animalis subsp. lactis strain DN-173 010 improved the symptoms of bloating and digestive discomfort, and reduced abdominal distension in patients with IBS-C (Guyonnet et al., Aliment Pharmacol Ther, 26, 475-86, 2007; Agrawal et al., Aliment Pharmacol Ther, 29, 104-114, 2008). The inventors have now found that the effect of Bifidobacterium lactis DN-173 010 on reduction of bloating is closely correlated with an increase in the intestinal population of Colinsella aerofaciens in the treated subjects.

Colinsella aerofaciens (formerly Eubacterium aerofaciens; Moore et al., Int J Syst Bacteriol, 21, 307-310, 1971; Kageyama et al., Int J Syst Bacteriol, 49, 557-565, 1999) belongs to the class of Actinobacteria.

It has been reported that a high population of Colinsella aerofaciens in the fecal flora is associated with a low risk of colon cancer (Moore & Moore, Appl Enviro. Microbiol., 61, 3202-3207, 1995). It has also been reported that Colinsella aerofaciens is less abundant in the faecal microbiota of IBS patients than in that of healthy subjects (Kassinen et al., 2007, cited above); however, no correlation between the amount of Colinsella aerofaciens and any of the symptoms of IBS has been shown until now.

Tannock et al. (Appl Enviro. Microbiol., 70, 2129-2136, 2004), also report that consumption of biscuits containing fructo- or galacto-oligosaccharides increased the metabolic activity of Bifidobacterium adolescentis and Colinsella aerofaciens in the faecal microbiota of healthy subjects, while having no effect on the sizes of the populations of these bacteria.

The inventors have found that other dietary fibres are able to increase the amount of C. aerofaciens in the faecal microbiotia. These are long chain inulin, retrograde resistant starch and galacturonic acid oligosaccharides.

An object of the present invention is a composition comprising a probiotic Bifidobacterium and/or a dietary fiber for increasing the population of C. aerofaciens in the intestinal microbiotia of a subject. Said subject is preferably a human subject; however it can also belong to another animal species, especially mammalian species, prone to diseases associated with a decrease in the intestinal population of Colinsella aerofaciens.

This composition is useful in particular for preventing or treating conditions associated with a low intestinal population of Colinsella aerofaciens. This includes in particular preventing or alleviating intestinal discomfort, for instance intestinal bloating and/or feeling of bloating, as well as decreasing the risk of occurrence of colon cancer.

The probiotic Bifidobacterium is typically selected among Bifidobacterium longum, Bifidobacterium breve, Bifidobacterium bifidum, Bifidobacterium infantis, Bifidobacterium lactis, Bifidobacterium animalis, Bifidobacterium animalis subsp. lactis, Bifidobacterium infantis, and Bifidobacterium adolescentis. Preferably, it belongs to the species Bifidobacterium animalis subsp. lactis. A particularly preferred Bifidobacterium animalis subsp. lactis strain is the strain DN-173 010 (CNCM 1-2494, described for instance in EP 1297176). In another embodiment, said probiotic Bifidobacterium is different from DN-173 010.

Preferably the dietary fiber is selected among long chain inulin, retrograde resistant starch and galacturonic acid oligosaccharides.

Long chain inulin is a mixture of fructans with an average degree of polymerization (DP) ranging from 20 to 25. It results from the removal of short-chain fructans (DP≦10) from standard inulin (which is mixture of fructans with DP ranging from 2 to 60, with an average DP of about 10).

Resistant starch is starch which is not digested in the small intestine and enters the large intestine. Retrograded resistant starch (also known as RS3 starch), is formed when starch which has been gelatinised by heating, is cooled. Retrogradation which occurs upon cooling converts part of the gelatinised starch to a crystalline form which is resistant to digestion.

Galacturonic acid oligosaccharides are oligosaccharides wherein at least 50 mol % of the monosaccharide units present in the oligosaccharide consist of galacturonic acid. The galacturonic acid oligosaccharides used in the invention are preferably prepared from degradation of pectin, pectate, and/or polygalacturonic acid. Preferably the degraded pectin is prepared by hydrolysis and/or beta-elimination of fruit and/or vegetable pectins, more preferably apple, citrus and/or sugar beet pectin, even more preferably apple, citrus and/or sugar beet pectin degraded by at least one lyase. In a preferred embodiment, at least one of the terminal galacturonic acid units of the galacturonic acid oligosaccharide has a double bond. The double bond effectively protects against attachment of pathogenic bacteria to intestinal epithelial cells. Preferably one of the terminal galacturonic acid units comprises a C4-05 double bond. The galacturonic acid oligosaccharide can be esterified, in particular methylated, acetylated and/or amidated. In a preferred embodiment the galacturonic acid oligosaccharides are methylated. Their degree of methylation is preferably from 20% to 70%, and more preferably from 30% to 50%. Methods for the manufacture of esterified pectin hydrolysates that can be suitably used in the present method and composition are provided in WO 01/60378 and/or WO 02/42484.

According to a particular embodiment, the compositions of the invention comprising a probiotic Bifidobacterium, or a dietary fiber or a mixture of both, further comprise bacteria of the species Colinsella aerofaciens.

Another object of the present invention is a composition comprising Colinsella aerofaciens for use for reducing intestinal bloating and/or the feeling of bloating, preferably for use in a subject suffering from irritable bowel syndrome, in particular from IBS-C.

Strains of Colinsella aerofaciens which are more particularly suitable for use in the compositions of present invention are non arthritogenic strains, which can easily be identified for instance on the basis of the sensitivity of their cell walls to enzymatic digestion with lysozyme or with mutanolysin (Zhang et al., Infect. Immun., 69, 7277-7284, 2001). In a preferred embodiment, a composition of the invention comprises from about 10³ CFU/ml to about 10¹¹ CFU/ml of Colinsella aerofaciens.

Examples of compositions of the invention are pharmaceutical or nutritional compositions. Nutritional compositions of the invention also include food products and in particular dairy products, as well as food supplements. A “food supplement” designates a product made from compounds usually used in foodstuffs, but which is in the form of tablets, powder, capsules, potion or any other form usually not associated with aliments, and which has beneficial effects for one's health.

The compositions of the invention can be in any form suitable for administration, in particular oral administration. This includes for instance solids, semi-solids, liquids, and powders. Liquid composition are generally preferred for easier administration, for instance as drinks.

The compositions of the invention may also comprise one or more strain(s) of lactic acid bacteria other than Bifidobacterium and Colinsella aerofaciens. For example, they may comprise one or more strain(s) of the following genera: Lactobacillus, Lactococcus, Streptococcus, Enterococcus and in particular of the following species: Lactobacillus acidophilus, Lactobacillus plantarum, Lactobacillus casei subsp. casei, Lactobacillus casei subsp. rhamnosus, Lactobacillus casei subsp paracasei, Lactobacillus lactis, Lactobacillus helveticus, Lactobacillus cremoris, Lactobacillus delbrueckii subsp. bulgaricus, Lactobacillus delbrueckii subsp. lactis, Lactobacillus delbrueckii subsp. delbrueckii, Lactobacillus reuteri, Lactobacillus amylovorus, Lactobacillus johnsonii, Lactobacillus fermentum, Lactobacillus brevis, Streptococcus thermophilus, Lactococcus lactis subsp. lactis, Lactococcus lactis subsp. cremoris.

The present invention also provides a method for selecting nutritional compounds able to increase the amount of Colinsella aerofaciens in the intestinal microbiotia of a mammal, preferably human, said method comprising the step of incubating a faecal sample from said mammal, under culture conditions suitable for growth of bacteria of the intestinal microflora, and in the presence of each nutritional compound to be tested, and determining the effect of said nutritional compound on the population of Colinsella aerofaciens in the culture.

Various methods for determining the population of Colinsella aerofaciens are known in themselves. Preferably, said population is determined by RT-qPCR.

According to a preferred embodiment of the method of the invention, the nutritional compounds to be screened are dietary fibres, lactic acid bacteria, or combinations thereof.

Preferably, the method of the invention is used for selecting compounds which can be used in a nutritional composition for treating and/or preventing intestinal bloating.

The present invention will be understood more clearly with the aid of the additional description which follows, which refers to non limiting examples illustrating the relation between the intestinal population of Colinsella aerofaciens and the reduction of bloating and/or of the feeling of bloating in subjects suffering from IBS-C, and the effect of various dietary fibres on the growth of Colinsella aerofaciens in the faecal microbiota.

### EXAMPLE 1

### Correlation Between Colinsella aerofaciens Population and Bloating Score in IBS-C Patients

A study on the effects on IBS symptomatology of 4 weeks consumption of a fermented milk product containing Bifidobacterium lactis DN-173 010 (test group) vs. a milk-based non-fermented dairy product without probiotics (control group) was conduced in 34 female patients (17 in each group) with IBS-C. The protocol and the results of this study are described in Agrawal et al., (Aliment Pharmacol Ther, 29, 104-114, 2008). One of the symptoms analysed in this study was abdominal bloating, evaluated by the subjects on a 0-5 scale. This symptom was found to be alleviated in the test group.

Along with the assessment of IBS symptomatology, stool samples were collected at baseline (Day 0) and following 4-week consumption period, for analysis of the faecal microbial population. The samples were stored in RNAlater® stabilization reagent until RNA isolation.

The Colinsella aerofaciens population in faecal samples was quantified by reverse transcription-quantitative PCR (RT-qPCR), according to the protocol described by Matsuda et al. (Appl. Environ. Microbiol.; 75, 1961-1969, 2009), using the following primers, derived from Colinsella aerofaciens ATCC 25986T;

(SEQ ID NO: 1)
Forward primer: CCCGACGGGAGGGGA
(SEQ ID NO: 2)
Reverse primer: CTTCTGCAGGTACAGTCTTGA

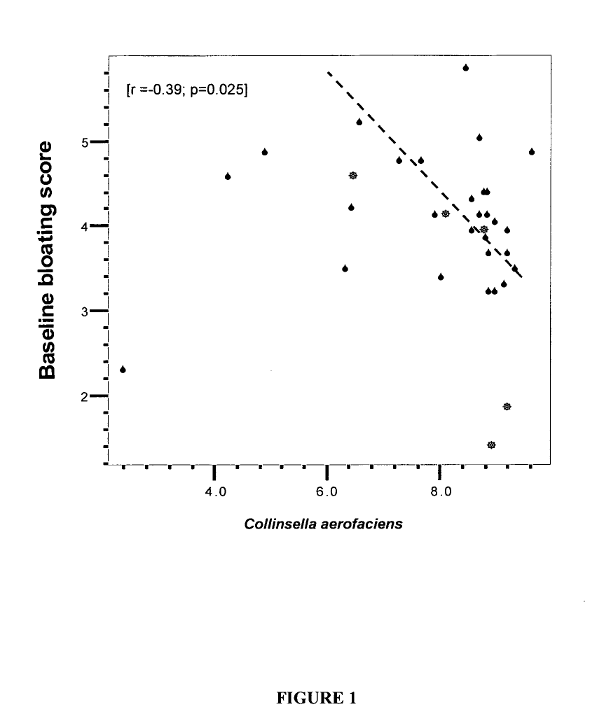

FIG. 1 represents the graph illustrating the relation at base line between the population of Colinsella aerofaciens (log₁₀cells/g) and the severity of the bloating symptoms (0-5 scale). This graph shows that the levels of Colinsella aerofaciens are inversely correlated with bloating symptoms severity (r=−0.39, p<0.01).

After the 4-week consumption period, the test group had significantly higher mean values of Colinsella aerofaciens compared with the control group (8.41 vs 7.55 log₁₀cells/g of faces, p=0.028, ANCOVA adjusted to baseline).

### EXAMPLE 2

### Effect of Dietary Fibres on the Amount of C. aerofaciens in the Faecal Microbiotia

An in vitro fermentation system was used using faeces from human adult. Fresh faecal material from 4 adult healthy donors was pooled and stored at −80° C. in the presence of 10% v/v glycerol. The faeces was thawed and mixed with McBain and McFarlane fermentation medium (Buffered peptone water 3.0 g/l, Yeast Extract 2.5 g/l, Tryptone 3.0 g/l, L-Cysteine-HCl 0.4 g/l, Bile salts 0.05 g/l, K₂HPO₄.3H2O 2.6 g/l, NaHCO₃ 0.2 g/l, NaCl 4.5 g/l, MgSO₄.7H₂O 0.5 g/l, CaCl_{2.}2H₂O 0.3 g/l, FeSO₄.7H₂O 0.005 g/l. Ingredients have to be added one by one in 800 ml water, adjust pH to 5.5±0.1 with K₂HPO₄ or NaHCO₃ and fill up to 1 litre.) which is representative for the intestinal environment, at a weight ratio 1:5.

At t=0, 6 ml of faecal suspension was mixed thoroughly with 200 mg of each fiber tested. As controls the faecal suspension was incubated without additive (blanc) or with 200 mg of glucose (glucose control). The faecal suspension was transferred into a dialysis tube in a 100 ml bottle with buffered dialysis medium (per litre: K₂HPO₄.3H₂O 2.6 g, NaHCO₃ 0.2 g, NaCl 4.5 g, MgSO₄.7H2O 0.5 g, CaCl₂.2H₂O 0.3 g, FeSO₄.7H₂O 0.005 g pH 6.3)). The bottle was closed and incubated at 37° C. Experiments were performed in duplo and all handlings were performed in an anaerobic cabinet.

Samples of 500 μl were taken at t=0 and t=48 h from the dialysis tube and resuspended in 1 ml RNA later™ (Ambion, Canada). 200 μl of the suspension was added to 1 ml PBS, centrifuged at 12.000 rpm for 5 minutes and pellets were stored at −80° C. until further use.

The following fibres were tested:

Raftiline GR (Orafti), which is a standard inulin with an average DP of about 10;
Frutalose L85 (Sensus), a hydrolyzed inulin with an average DP of about 4;
Actilight (Beghin Meiji), a fructo-oligosaccharide with an average DP of between 3 and 4;
RaftilinHP (which is an inulin from which the shorter chains have been removed and which typically has an average DP between 20 and 25);
Vivinal GOS (Friesland Foods DOMO), transgalacto-oligosaccharides with a DP below 6;
Novelose 330 (National starch), resistant maize starch of the retrograded, RS3 type;
Actistar (Cargill), resistant tapioca starch of the retrograded RS3 type;
Fibersol 2 (Matsutani), resistant maltodextrin with an average molecular mass of 2000 kDa, and with linkages in the molecule randomly distributed among units consisting of α- and β- (1→4), (1→6), (1→2), and (1→3) glycosidic bonds);
Nutriose FB06 (Roquette), resistant maltodextrin with branched chains and with a weight average molecular weight of about 5000 g/mol, in addition to alpha 1,4 linkages 30% a 1,5 10% alpha 1,2 and 10% alpha 1,3 linkages;
STA lite polydextrose (Tate & Lyle), resistant maltodextrin with average DP 9-10;
pectin derived acidic oligosaccharides (AOS) (Südzucker) a pectin hydrolysate with a degree of methylation of about 40%, an average degree of polymerization between 1 to 20, and which consists of approximately 75% galacturonic acid oligomers;
SSPS (Fuji oil), soluble soy polysaccharides;
xanthan gum (CP Kelco);
dextran (Sigma);
HM pectin (CP Kelco) a high molecular weight pectin with a degree of methylation above 50%;
Gum acacia (CNI);

### Gum Tragacanth (Willy Benecke);

PHGG (partially hydrolyzed guar gum).

Faeces was analyzed and the amount of C. aerofaciens was determined according to Example 1.

The results are shown in Table 1 below. Surprisingly only specific tested fibres were able to increase the amount of C. aerofaciens in the faecal microbiotia after 48 h compared with the blanc and compared with the glucose control. These fibres were long chain inulin, resistant starch and galacturonic acid oligosaccharides. The inulin chain has to be long in order to have the best C. aerofaciens promoting effect, since inulin with a standard DP of about 10 and short chain inulin with a DP of 3 to 4 was not able to increase C. aerofaciens significantly. Also resistant starch was able to increase C. aerofaciens. Since resistant dextrin was not able to induce C. aerofaciens significantly, the structure of resistant starch has to be of the retrograded or RS3 type. The other fiber able to induce C. aerofaciens turned out to be AOS, a pectin hydrolysate. Since pectin HM was less efficient, the pectin needs to be an oligosaccharide, preferably with an average degree of polymerization below 100, more preferably below 50, even more preferably below 20.

TABLE 1
Effect of fibres on C. aerofaciens levels in human faeces

| | | C. aerofaciens |
|---|---|---|
| | Additive | (log/g faeces) |
| t = 0 | | 9.29 |
| t = 48 h | None (Blanc) | 9.06 |
| | Glucose | 9.52 |
| | Raftiline GR | 9.60 |
| | Frutalose L85 | 9.19 |
| | Actilight | 9.47 |
| | RaftilinHP | 9.79* |
| | Vivinal GOS | 9.10 |
| | Novelose 330 | 9.70* |
| | Actistar | 9.87* |
| | Fibersol 2 | 9.40 |
| | Nutriose FB 06 | 9.46 |
| | STA lite polydextrose | 9.36 |
| | SSPS | 9.17 |
| | AOS | 10.19* |
| | HM pectin | 8.81 |
| | Dextran | 8.81 |
| | Xanthan gum | 8.64 |
| | Gum acacia | 9.08 |
| | Gum tragacanth | 9.04 |
| | PHGG | 9.46 |

*P < 0.05

1. A composition comprising an effective amount of a dietary fiber selected from retrograde resistant starch, galacturonic acid oligosaccharides, a probiotic Bifidobacterium, or combinations thereof for increasing the population of C. aerofaciens in the intestinal microbiotia in a subject.

2. The composition of claim 1, wherein the Bifidobacterium is a Bifidobacterium animalis subsp. lactis strain CNCM I-2494.

3. The composition of claim 1, further comprising Colinsella aerofaciens.

4. The composition of claim 1, wherein the composition prevents or reduces intestinal bloating.

5. The composition of claim 1, wherein the subject is suffering from irritable bowel syndrome (IBS).

6. The composition of claim 3, wherein the Collinsella aerofaciens is in a concentration range of about 10³ CFU/ml to about 10¹¹ CFU/ml.

7. A method of increasing the population of C. aerofaciens in the intestinal flora comprising administering to the subject in need thereof, a therapeutically effective amount of the composition of claim 1.

8. The method of claim 7, wherein the composition further comprises Colinsella aerofaciens.

9. The method of claim 7, wherein the subject is suffering from IBS.

10. The method of claim 9, wherein the IBS is IBS with constipation (IBS-C).

11. A method of preventing or reducing intestinal bloating comprising administering to a subject in need thereof a therapeutically effective amount of the composition of claim 1.

12. The method of claim 11, wherein the composition further comprises Colinsella aerofaciens.

13. The method of claim 11, wherein the subject is suffering from IBS.

14. The method of claim 13, wherein the IBS is IBS-C.


SEQUENCE LISTING (2 sequences)

SEQ ID NO: 1 - DNA, 15 bp, Collinsella aerofaciens
cccgacgggagggga

SEQ ID NO: 2 - DNA, 21 bp, Collinsella aerofaciens
cttctgcaggtacagtcttga